(19) United States
(12) Patent Application Publication
(10) Pub. No.: US 2024/0290470 A1
(43) Pub. Date: Aug. 29, 2024

(54) MEDICAL INFORMATION PROCESSING APPARATUS AND MEDICAL INFORMATION PROCESSING SYSTEM

(71) Applicant: CANON MEDICAL SYSTEMS CORPORATION, Tochigi (JP)

(72) Inventors: Tomoyuki OYAMA, Otawara (JP); Shinya NISHITAKA, Nasushiobara (JP); Shiro KATO, Utsunomiya (JP); Kyohei NORO, Nasushiobara (JP); Yuto TAKAHASHI, Otawara (JP); Guang yi ONG, Nasushiobara (JP)

(73) Assignee: CANON MEDICAL SYSTEMS CORPORATION, Tochigi (JP)

(21) Appl. No.: 18/402,882

(22) Filed: Jan. 3, 2024

(30) Foreign Application Priority Data
Feb. 27, 2023 (JP) 2023-028669

(51) Int. Cl. G16H 30/40; G06T 15/00; G16H 30/20

(52) U.S. Cl. CPC G16H 30/40; G06T 15/00; G16H 30/20

(57) ABSTRACT

A medical information processing apparatus according to an embodiment includes a processing circuitry. In the medical information processing apparatus according to the embodiment, first, the processing circuitry acquires an analysis result of each of a plurality of algorithms for a medical image. The processing circuitry causes a terminal of a user to display a screen in a display mode combining the analysis results of the algorithms. Thus, by the medical information processing apparatus according to the embodiment, the efficiency of image reading can be improved.

### CROSS-REFERENCE TO RELATED APPLICATIONS

This application is based upon and claims the benefit of priority from Japanese Patent Application No. 2023-028669, filed on Feb. 27, 2023; the entire contents of which are incorporated herein by reference.

### FIELD

Embodiments disclosed in the present specification and drawings generally relate to a medical information processing apparatus and a medical information processing system.

### BACKGROUND

In recent years, in imaged-based diagnosis, more physicians have come to read medical images by referring to the analysis results of artificial intelligence (AI) algorithms (especially deep learning) for the medical images. Here, if just a single algorithm is used, it may fail to obtain the analysis results that are useful for reading images of diseases. In view of this, using a plurality of algorithms may be able to support the image reading. However, when a physician refers to the analysis result of each of the algorithms, the physician may spend time looking for the range to read the image, which may fail to improve the efficiency of the image reading.

### BRIEF DESCRIPTION OF THE DRAWINGS

FIG. 1 is a diagram illustrating an example of a structure of a medical information display system including a medical information processing apparatus according to an embodiment;

FIG. 2 is a diagram illustrating an example of a structure of the medical information processing apparatus according to the embodiment;

FIG. 3 is a flowchart expressing a procedure of a process by the medical information processing apparatus according to the embodiment;

FIG. 4 is a diagram for describing a scoring algorithm;

FIG. 5 illustrates an example of a screen displayed on a terminal (first display example);

FIG. 6 illustrates an example of the screen displayed on the terminal (second display example);

FIG. 7 illustrates an example of the screen displayed on the terminal (third display example);

FIG. 8 illustrates an example of the screen displayed on the terminal (fourth display example); and

FIG. 9 illustrates another example of the screen displayed on the terminal.

### DETAILED DESCRIPTION

A medical information processing apparatus according to an embodiment includes a processing circuitry. The processing circuitry acquires an analysis result of each of a plurality of algorithms for a medical image. The processing circuitry causes a terminal of a user to display a screen in a display mode combining the analysis results of the algorithms.

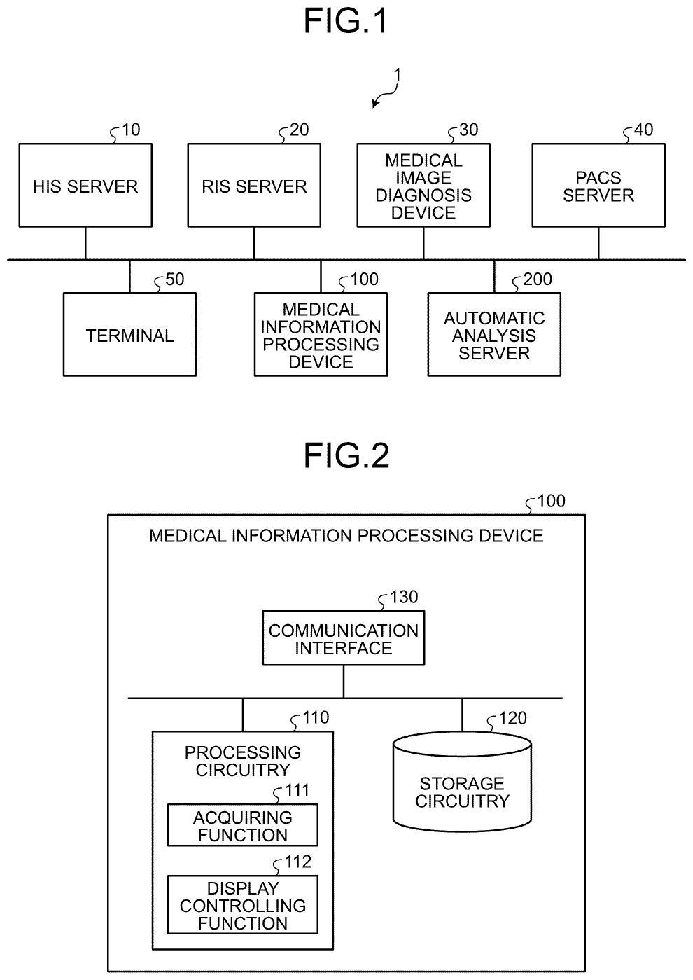

One embodiment of the medical information processing apparatus is described below in detail with reference to the drawings. Note that in the example to be described below, a medical information processing system includes the medical information processing apparatus. In a medical information processing system illustrated in FIG. 1, the respective apparatuses are illustrated one by one but in reality, more apparatuses can be arranged.

A medical information processing system 1 illustrated in FIG. 1 includes, for example, a hospital information system (HIS), a radiology information system (RIS), and a picture archiving and communication system (PACS). The medical information processing system 1 includes an HIS server 10, an RIS server 20, a medical image diagnosis apparatus 30, a PACS server 40, a terminal 50, a medical information processing apparatus 100, and an automatic analysis server 200.

The HIS server 10, the RIS server 20, the medical image diagnosis apparatus 30, the PACS server 40, the terminal 50, the medical information processing apparatus 100, and the automatic analysis server 200 are connected to, for example, an in-hospital local area network (LAN) installed in a hospital, send information to a predetermined apparatus, and receive information sent from the predetermined apparatus. The HIS server 10 may be connected to an external network in addition to the in-hospital LAN. The automatic analysis server 200 may be located in an external network or in the cloud.

For example, the terminal 50 is used by a user involved in the medical treatment of a patient. Examples of the user include medical workers such as a physician and a radiologist. The terminal 50 includes, for example, a personal computer (PC), a tablet PC, a personal digital assistant (PDA), and a mobile terminal.

In the HIS, the HIS server 10 illustrated in FIG. 1 manages information generated in the hospital. The information generated in the hospital includes patient information, examination order information, and the like.

The patient information includes basic information, medical treatment information, and examination implementation information of the patient. The basic information includes patient ID, name, date of birth, gender, blood type, height, weight, and the like. As the patient ID, identifier information that uniquely identifies the patient is set. The medical treatment information of the patient includes information such as numerical values (measurement values) and medical treatment records, as well as information indicating the date and time of the recording. For example, the medical treatment information of the patient includes prescriptions for medicines by physicians, nursing records by nurses, examinations in the laboratory department, and arrangements for meals during hospitalization. For example, the prescriptions are recorded in the electronic medical records by the physicians, and the nursing records are recorded in the electronic medical records by the nurses. The examination implementation information includes information on examinations conducted in the past and the examination results of such examinations, as well as information indicating the dates of implementation of those examinations.

The examination order information is issued to generate the examination implementation information. The examination order information includes examination ID, patient ID, an examination code, a clinical department, an examination type, an examination site, and scheduled examination date and time, etc. The examination ID is an identifier to uniquely identify the examination order information. The examination code is an identifier that uniquely identifies the examination. The clinical department indicates the specialty category of the medical treatment. The examination type indicates an examination using medical images. Examples of the examination type include an X-ray examination, a computed tomography (CT) examination, and a magnetic resonance imaging (MRI) examination. The examination sites include the brain, kidneys, lungs, liver, bones, and the like.

When the examination order information is input from an examination request physician, for example, the HIS server 10 sends the input examination order information and the patient information specified by the examination order information to the RIS. In this case, the HIS server 10 also sends the patient information to the PACS.

In the RIS, the RIS server 20 illustrated in FIG. 1 manages the information related to radiological examination services. For example, the RIS server 20 receives the examination order information sent from the HIS server 10, adds various setting information to the received examination order information, accumulates the information, and manages the accumulated information as examination reservation information. Specifically, upon the reception of the patient information and the examination order information sent from the HIS server 10, the RIS server 20 generates the examination reservation information necessary to operate the medical image diagnosis apparatus 30, based on the received patient information and examination order information. The examination reservation information includes information necessary to perform the examination, for example, the examination ID, the patient ID, the examination type, and the examination site. The RIS server 20 sends the generated examination reservation information to the medical image diagnosis apparatus 30.

The medical image diagnosis apparatus 30 illustrated in FIG. 1 is an apparatus by which a clinical technologist performs an examination by photographing a patient, for example. Examples of the medical image diagnosis apparatus 30 include an ultrasonic diagnosis apparatus, an X-ray computed tomography (CT) apparatus, a magnetic resonance imaging (MRI) apparatus, a single photon emission computed tomography (SPECT) apparatus, a positron emission computed tomography (PET) apparatus, a SPECT-CT apparatus that combines the SPECT apparatus and the X-ray CT apparatus, and a PET-CT apparatus that combines the PET apparatus and the X-ray CT apparatus. The medical image diagnosis apparatus 30 is also referred to as a modality apparatus.

The medical image diagnosis apparatus 30 performs examinations on the basis of the examination reservation information sent from the RIS server 20, for example. The medical image diagnosis apparatus 30 then generates the examination implementation information representing the implementation of the examination, and sends the examination implementation information to the RIS server 20. In this case, the RIS server 20 receives the examination implementation information from the medical image diagnosis apparatus 30 and outputs the received examination implementation information to the HIS server 10 as the latest examination implementation information. For example, the HIS server 10 receives the latest examination implementation information and manages the received examination implementation information. The examination implementation information includes the examination reservation information (examination ID, patient ID, examination type, examination site, etc.), the date and time of the examination, and the like.

In addition, the medical image diagnosis apparatus 30 generates medical image data when a clinical technologist photographs a subject (patient) in the implementation of the examination. Examples of the medical image data include X-ray CT image data, X-ray image data, MRI image data, nuclear medicine image data, and ultrasonic image data. The medical image diagnosis apparatus 30 converts the generated medical image data into a format compliant with the Digital Imaging and Communication in Medicine (DICOM) standard, for example. In other words, the medical image diagnosis apparatus 30 generates medical image data to which DICOM tags are added as supplementary information. The medical image diagnosis apparatus 30 sends the generated medical image data to the PACS.

The supplementary information includes, for example, patient ID, examination ID, apparatus ID, image series ID, conditions related to the photographing, etc., and is standardized according to the DICOM standard. The apparatus ID is information to identify the medical image diagnosis apparatus 30. The image series ID is information to identify single photographing by the medical image diagnosis apparatus 30 and includes, for example, the photographed part of the subject (patient), the time of image generation, slice thickness, and a slice position. For example, by performing the CT examination or the MRI examination, a tomographic image (slice image) at each of a plurality of slice positions is obtained as the medical image data.

In the PACS, the PACS server 40 illustrated in FIG. 1, for example, receives the patient information sent from the HIS server 10 and manages the received patient information. The PACS server 40 includes a storage circuitry for managing the patient information. For example, the PACS server 40 receives the medical image data sent from the medical image diagnosis apparatus 30, and stores the received medical image data in its own storage circuitry by associating the medical image data with the patient information. The PACS server 40, for example, reads out the medical image data from its own storage circuitry in response to an acquisition request from the medical information processing apparatus 100 and sends the medical image data to the medical information processing apparatus 100. The supplementary information such as patient ID, examination ID, apparatus ID, and image series ID is added to the medical image data saved in the PACS server 40.

Therefore, the user can acquire the necessary patient information from the PACS server 40 by performing a search using patient ID or the like. The user can acquire the necessary medical image data from the PACS server 40 by performing a search using patient ID, examination ID, apparatus ID, image series ID, or the like.

The automatic analysis server 200 has a plurality of algorithms and outputs the analysis result of each of the algorithms for the medical image data (hereinafter referred to as medical image) stored in the PACS server 40. Each of the algorithms may be an artificial intelligence (AI)-based algorithm or an algorithm that is not AI based. The algorithms and the analysis result of each of the algorithms are discussed below.

The medical information processing apparatus 100 illustrated in FIG. 1 is a workstation to cause a display of the terminal 50 to display the medical treatment information, the medical images, and the like of the patient as the information related to the medical treatment of the patient.

The medical information processing apparatus 100 according to this embodiment will hereinafter be described in detail. FIG. 2 is a diagram illustrating an example of a structure of the medical information processing apparatus 100 according to this embodiment. As illustrated in FIG. 2, the medical information processing apparatus 100 includes a processing circuitry 110, a storage circuitry 120, and a communication interface 130.

The storage circuitry 120 is connected to the processing circuitry 110 and stores various kinds of information therein. Specifically, the storage circuitry 120 stores therein the patient information received from each system. For example, the storage circuitry 120 is realized by a semiconductor memory element such as a random-access memory (RAM) or a flash memory, a hard disk, an optical disc, or the like. Here, the storage circuitry 120 is an example of a storage unit. The communication interface 130 is, for example, a network interface card (NIC), which communicates with other apparatuses.

The processing circuitry 110 controls components of the medical information processing apparatus 100. For example, the processing circuitry 110 performs an acquiring function 111 and a display controlling function 112 as illustrated in FIG. 2. Here, for example, each processing function performed by the acquiring function 111 and the display controlling function 112, which are the components of the processing circuitry 110, is recorded in the storage circuitry 120 in the form of a computer program executable by a computer. The processing circuitry 110 is a processor that reads out each computer program from the storage circuitry 120 and executes the computer program to realize the function corresponding to the computer program. In other words, the processing circuitry 110 that has read out each computer program has each function illustrated in the processing circuitry 110 in FIG. 2.

The term “processor” used in the above description means a circuitry such as a central processing unit (CPU), a graphics processing unit (GPU), or an application specific integrated circuit (ASIC). The term “processor” also means a circuitry such as a programmable logic device. Examples of the programmable logic device include a simple programmable logic device (SPLD) and a complex programmable logic device (CPLD). Other examples of the programmable logic device include a field programmable gate array (FPGA). If the processor is a CPU, for example, the processor reads out and executes a computer program saved in the storage circuitry 120 to realize the function. On the other hand, if the processor is an ASIC, for example, the computer program is incorporated directly into the circuitry of the processor instead of saving the computer program in the storage circuitry 120. Each processor in this embodiment is not limited to the case where each processor is configured as a single circuit, but may also be configured as a single processor by combining a plurality of independent circuits to realize the functions. Furthermore, a plurality of components in FIG. 2 may be integrated into a single processor to achieve the function.

In recent years, in the image-based diagnosis, for example, more physicians have come to read medical images with reference to the analysis results of AI algorithms (especially deep learning) for the medical images. Here, if just a single algorithm is used, it may fail to obtain the analysis results that are useful for reading images of diseases. In view of this, using a plurality of algorithms may be able to support the image reading. However, when a physician refers to the analysis result of each of the algorithms, the physician may spend time looking for the range to read the image, which may fail to improve the efficiency of the image reading.

Therefore, the medical information processing apparatus 100 in this embodiment performs the following processes to improve the efficiency of the image reading. First, in the medical information processing apparatus 100 according to this embodiment, the acquiring function 111 acquires the analysis result of each of the algorithms for the medical images. The display controlling function 112 causes the terminal 50 of the user, who is the medical worker such as a physician or a radiologist, to display a screen in a display mode combining the analysis results of the multiple algorithms. Here, the acquiring function 111 and the display controlling function 112 are examples of an acquiring unit and a display controlling unit, respectively.

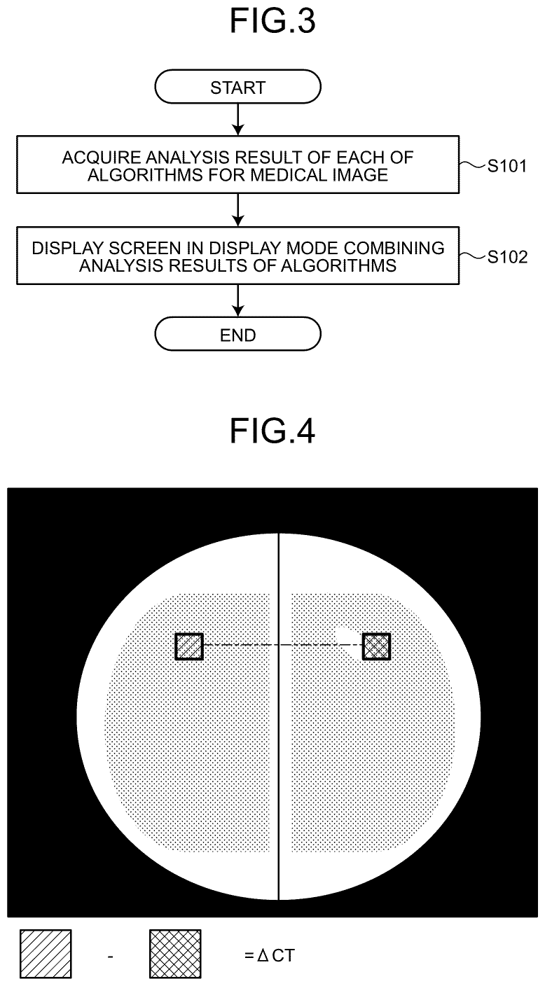

First, a process of the medical information processing apparatus 100 according to this embodiment will be described. FIG. 3 is a flowchart expressing the procedure of the process by the medical information processing apparatus 100 according to this embodiment.

At step S101 in FIG. 3, the processing circuitry 110 calls a computer program corresponding to the acquiring function 111 from the storage circuitry 120 and executes the computer program. At step S101, the acquiring function 111 acquires the analysis result of each of the algorithms for the medical image from the automatic analysis server 200. For example, the medical image is a three-dimensional image formed of a plurality of slice images. For example, when analyzing intracerebral hemorrhage, a slice image in a region with left-right symmetry will show a left-right difference in an axial image of a head. The following is a description of an example of a plurality of algorithms for analyzing intracerebral hemorrhage using the axial images of the head (hereinafter simply referred to as “multiple algorithms”) and the analysis result of each of the multiple algorithms.

The analysis of the image in the region with the left-right symmetry is used, for example, for rapid diagnosis in emergency situations, but may also be used to analyze changes over time between the past image and the present image. Regarding the analysis of the image in the region with the left-right symmetry, the case in which the axial images are used is described as an example in this embodiment; however, the embodiment is not limited to this example, and sagittal or coronal images may also be used depending on the analysis.

The multiple algorithms include, for example, an algorithm to score CT values of the slice images (hereinafter referred to as algorithm A) and an algorithm to score left-right differences in the shape of the region with the left-right symmetry of the slice images (hereinafter referred to as algorithm B).

First, the analysis result of the algorithm A is described. In the slice images, the algorithm A divides each slice image into a plurality of regions and scores the CT value of each slice image. Specifically, in each slice image, the algorithm A performs an analysis in which a region with a small difference in CT value is given a low score and a region with a large difference in CT value is given a high score. Here, the score for each slice image is derived by finding the average of the sum of the differences of the respective divided regions in a single slice image. The algorithm A then derives the analysis result of normalizing the score for each of the slice images in the range of 0 to 100.

Here, the scoring algorithm is described as the analysis result of the algorithm A. FIG. 4 is a diagram for describing the scoring algorithm. FIG. 4 illustrates a slice image of a brain in a case of providing a line for cutting the slice image into a half vertically as a reference line for taking the left-right difference in CT value. Here, the reference line may be the midline. For example, the scoring algorithm divides each of the slice images of the brain into a plurality of regions and scores the CT value in each of the slice images. Specifically, the scoring algorithm calculates the difference ΔCT between the left and right CT values for each region in each slice image, using the formula shown below.

$$Δ C {T}_{Ave , k} = \frac{{\sum }_{n = 1}^{All pixel num \times \frac{1}{2}} Δ C {T}_{n}}{All pixel num \times \frac{1}{2}}$$

Here, n is the number of divided regions, which is expressed by n=1, 2, . . . , n. Additionally, k is the number of slice images, which is expressed by k=1, 2, . . . , k. The scoring algorithm then calculates the average value ΔCT_{Ave,1}, ΔCT_{Ave,2}, . . . , ΔCT_{Ave,k}, which is the average of the total of the difference ΔCT calculated for each region for the number k of slice images, and derives the analysis results of normalizing the score for each of the slice images in the range of 0 to 100.

If the analysis results can be derived by normalizing the scores for the respective slice images, the score for each slice image does not have to be derived by the average of the sum of the differences, and for example, may be derived by the sum of the differences.

Next, the analysis result of the algorithm B is described. When there is a difference between the left and right regions in the slice images, the algorithm B generates a pseudo image on the basis of each region using a generative adversarial network (GAN) and by taking the difference from the region on the opposite side, scores the left-right difference in shape of the region with the left-right symmetry in each slice image. The algorithm A then derives the analysis results of normalizing the scores of the slice images in the range of 0 to 100.

If the analysis results of normalizing the scores of the slice images can be derived, the score of each slice image may be derived by inverting the normal region in the left-right direction and taking the difference from the abnormal region instead of using GAN.

At step S102 in FIG. 3, the processing circuitry 110 calls a computer program corresponding to the display controlling function 112 from the storage circuitry 120 and executes the computer program. At step S102, the display controlling function 112 causes the terminal 50 of the user to display a screen in a display mode combining the analysis results of the multiple algorithms. The screen displayed on the terminal 50 will be described based on some display examples.

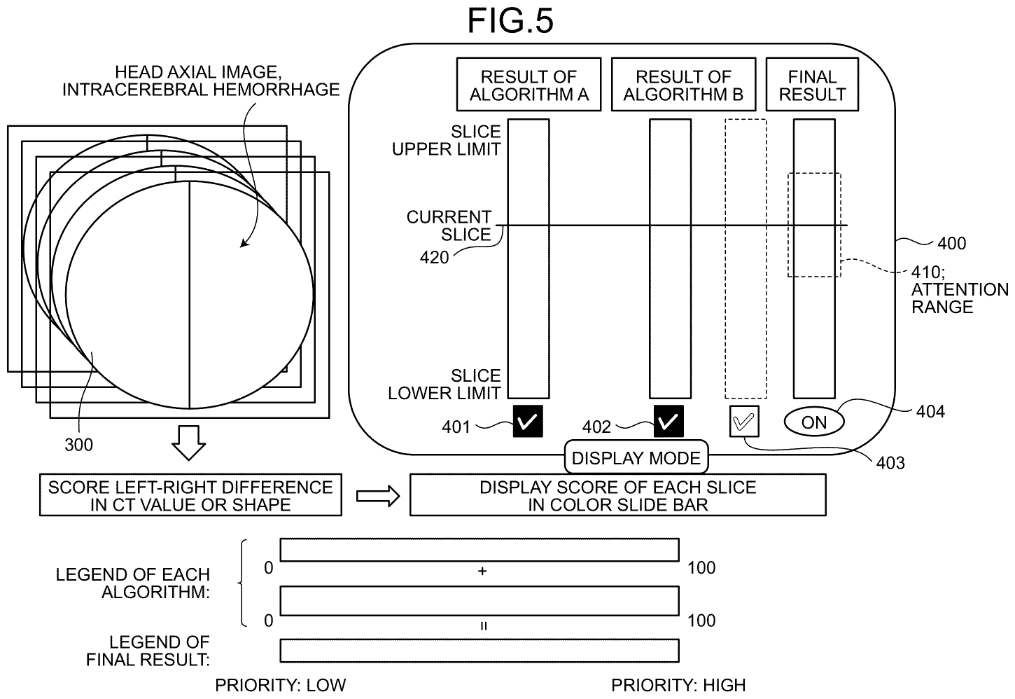

FIG. 5 illustrates an example of a screen 400 displayed on the terminal 50 (first display example). As illustrated in FIG. 5, the display controlling function 112 causes the terminal 50 of the user to display the screen 400 in the display mode combining the analysis results of the multiple algorithms.

The screen 400 displays a plurality of selection areas for selecting the analysis results of the multiple algorithms. Specifically, if the multiple algorithms are the algorithms A and B, the screen 400 displays selection areas 401 and 402 for selecting the analysis results of the algorithms A and B, respectively. In the example illustrated in FIG. 5, when the user selects the selection areas 401 and 402 on the terminal 50, the display controlling function 112 causes the terminal 50 to display the screen 400 in the display mode combining the analysis results of the algorithms A and B selected by the user.

On the screen 400, the slice images are arranged in the body axis direction of the subject as the analysis results of the algorithms A and B, and for the analysis results of the algorithms A and B, the scores derived by the aforementioned deriving method are displayed in color slide bars as the scores in which the respective slice images are normalized in the range of 0 to 100. Specifically, as the score displayed on the screen 400 is closer to 0, that is, as the difference is smaller, the score is displayed in, for example, blue (in FIG. 5, the blue color is represented by an oblique hatching pattern). On the other hand, as the score displayed on the screen 400 is closer to 100, that is, as the difference is larger, the score is displayed in, for example, red (in FIG. 5, the red color is represented by a cross hatching pattern). The color slide bar gradually changes from blue to red as the score increases from 0 to 100. Thus, the display controlling function 112 causes the terminal 50 of the user to display the screen 400 in the display mode using the colors according to the scores indicating the analysis results of the multiple algorithms. As for the color of the color slide bar, FIG. 5 illustrates an example of the gradation using two colors; however, a single-color gradation, a multiple-color gradation, or mixed colors may be used alternatively.

Here, the display controlling function 112 causes the screen 400 to display the analysis results of the two algorithms A and B; however, the screen 400 may display any operation result among an OR operation result, an AND operation result, and an XOR operation result as the display mode combining the analysis results of two or more algorithms. For example, if the screen 400 displays the selection areas 401, 402, and 403 to select the analysis results of the algorithms A, B, and C, respectively and the user selects the selection areas 401, 402, and 403, the screen 400 displays any operation result among the OR operation result, the AND operation result, and the XOR operation result as the display mode combining the analysis results of the algorithms A, B, and C.

In the example illustrated in FIG. 5, the screen 400 displays the OR operation result as the display mode (final result) combining the analysis results of the algorithms A and B. For example, as the score of the OR operation result is closer to 0, the score is displayed in darker blue. This indicates that the priority for the image reading is low. On the other hand, as the score of the OR operation result is closer to 100, the score is displayed in darker red. This indicates that the priority for the image reading is high. In the example illustrated in FIG. 5, the range displayed in red by the OR operation result is displayed as the range of the slice images to which attention needs to be paid in the slice images, that is, as an attention range 410. Thus, the display controlling function 112 causes the terminal 50 of the user to display on the screen 400, the range of the slice images to which attention needs to be paid in the slice images, based on the analysis results of the multiple algorithms.

The screen 400 also displays a button 404 to enable switching between display and non-display of the display mode (final result) combining the analysis results of the algorithms A and B. For example, when the user presses the button 404, the final result is displayed on the screen 400; when the user presses the button 404 again, the final result is not displayed on the screen 400. In the example illustrated in FIG. 5, the final result is displayed on the screen 400 because the user presses the button 404 until “ON” appears on the button 404. When the user then presses the button 404 again, the button 404 displays “OFF” and the final result is not displayed on the screen 400.

In the example illustrated in FIG. 5, the display controlling function 112 causes the screen 400 to display the analysis results of the two algorithms A and B and the operation results combining the analysis results of the algorithms A and B; however, the screen 400 may display only the operation results.

As illustrated in FIG. 5, the screen 400 also displays a display designation bar 420 as a tool for the user to designate one of the slice images. For example, the user designates one slice image in the attention range 410 using the display designation bar 420 on the terminal 50. In this case, the display controlling function 112 performs the display of a slice image 300 as the current slice image together with the screen 400, the slice image 300 being designated by the user with the display designation bar 420. In the example illustrated in FIG. 5, referring to the slice image 300, that is, the axial image of the head, it can be confirmed that there is bleeding in the brain.

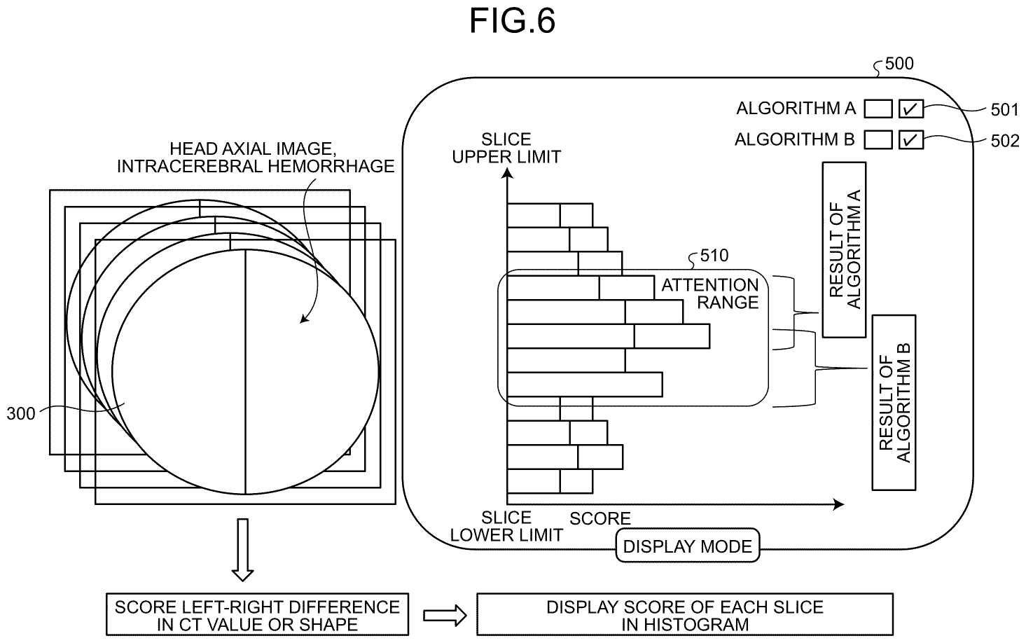

FIG. 6 illustrates an example of a screen 500 displayed on the terminal 50 (second display example). As illustrated in FIG. 6, the display controlling function 112 causes the terminal 50 of the user to display the screen 500 in the display mode combining the analysis results of the multiple algorithms.

The screen 500 displays a plurality of selection areas for selecting the analysis results of the multiple algorithms. Specifically, in the example illustrated in FIG. 6, when the user selects selection areas 501 and 502 on the terminal 50, the display controlling function 112 causes the terminal 50 to display the screen 500 in the display mode combining the analysis results of the algorithms A and B selected by the user.

The display controlling function 112 causes the terminal 50 of the user to display on the screen 500, the range of the slice images to which attention needs to be paid in the slice images, based on the analysis results of the multiple algorithms. Specifically, the display controlling function 112 causes the terminal 50 to display the screen 500 in the display mode using a graph (histogram) in which the body axis direction of the subject and the scores indicating the analysis results of the multiple algorithms are associated. The vertical axis of the screen 500 indicates the body axis direction of the subject and the horizontal axis of the screen 500 indicates the scores of the multiple algorithms.

In the example illustrated in FIG. 6, the screen 400 displays the result of addition as the display mode (final result) combining the analysis results of the algorithms A and B. This indicates that the priority for the image reading is lower as the score of the result of addition is smaller. On the other hand, this indicates that the priority for the image reading is higher as the score of the result of addition is larger. In the example illustrated in FIG. 6, the range where the score of the result of addition is large is displayed as the range of the slice images to which attention needs to be paid in the slice images, i.e., an attention range 510. Here, in the example illustrated in FIG. 6, there is a slice image that contains only the score of the algorithm B in the attention range 510.

In the example illustrated in FIG. 6, the display designation bar 420 is omitted; however, the slice image 300 designated by the user is displayed on the terminal 50 of the user along with the screen 500 as the current slice image.

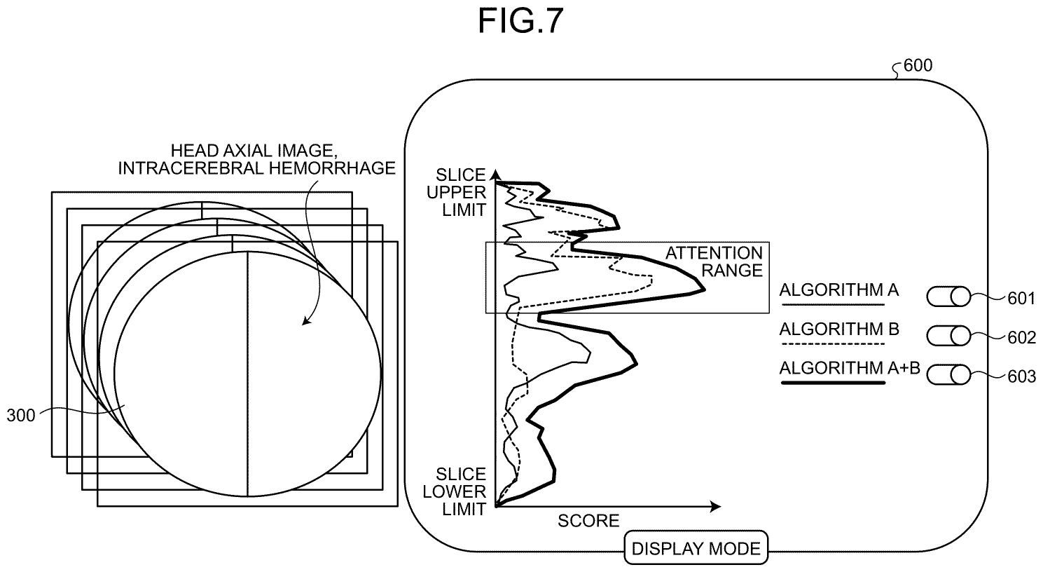

FIG. 7 illustrates an example of a screen 600 displayed on the terminal 50 (third display example). FIG. 7 illustrates an example of the screen 600 displayed on the terminal 50 in a display mode of a graph (e.g., line graph), which is different from that in FIG. 6.

The screen 600 displays a plurality of selection areas for selecting the analysis results of the multiple algorithms. Specifically, in the example illustrated in FIG. 7, when the user selects selection areas 601 and 602 on the terminal 50, the terminal 50 displays the screen 600 in the display mode combining the analysis results of the algorithms A and B. Furthermore, when the user selects a selection area 603 on the terminal 50, the screen 600 displays the result of addition as the display mode combining the analysis results of the algorithms A and B (algorithms A+B).

In the example illustrated in FIG. 7, the display designation bar 420 is omitted; however, the slice image 300 designated by the user is displayed on the terminal 50 of the user along with the screen 600 as the current slice image.

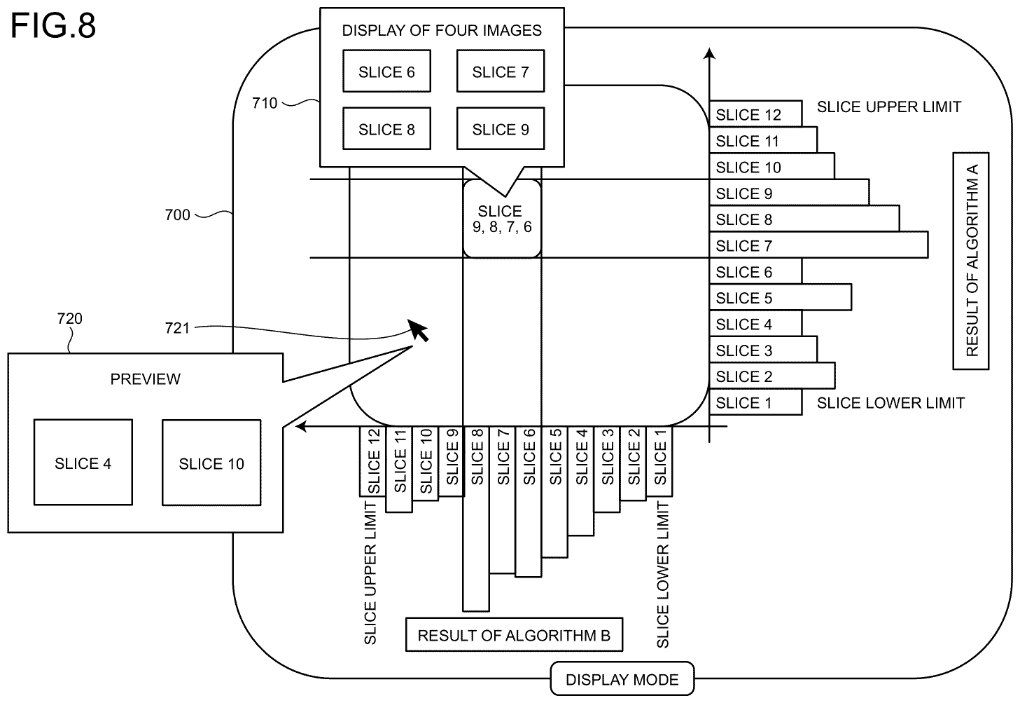

FIG. 8 illustrates an example of a screen 700 displayed on the terminal 50 (fourth display example). As illustrated in FIG. 8, the display controlling function 112 causes the terminal 50 of the user to display the screen 700 in the display mode combining the analysis results of the multiple algorithms.

Specifically, the display controlling function 112 causes the terminal 50 to display the screen 700 in a display mode using a heat map in which the score indicating the analysis result of the first algorithm (algorithm A) among the multiple algorithms and the score indicating the analysis result of the second algorithm (algorithm B) among the multiple algorithms are associated. The vertical axis of the screen 700 indicates the score of the algorithm A, and the horizontal axis of the screen 700 indicates the score of the algorithm B.

In the example illustrated in FIG. 8, the range displayed by the XOR operation result is displayed as the range of the slice images to which attention needs to be paid in the slice images, that is, an attention range 710. For example, in FIG. 8, the range of four slice images including the overlapped part between slice images “slices 7-9” as the analysis result of the algorithm A and slice images “slices 6-8” as the analysis result of the algorithm B is displayed as the attention range 710. Thus, the display controlling function 112 causes the terminal 50 of the user to display on the screen 700, the range of the slice images to which attention needs to be paid in the slice images, based on the analysis results of the multiple algorithms.

In the example illustrated in FIG. 8, the portion designated by the user with a mouse cursor 721 on the terminal 50 is displayed as the slice image to which the user pays attention. For example, in FIG. 8, two slice images including a slice image “slice 4” as the analysis result of the algorithm A and a slice image “slice 10” as the analysis result of the algorithm B are displayed as a preview 720.

Although the analysis results of the algorithms A and B are displayed in the display mode using the heat map, the display mode is not limited to the display mode using the heat map as long as the analysis results of the algorithms A and B are displayed together.

As described above, in the medical information processing apparatus 100 according to this embodiment, the acquiring function 111 acquires the analysis results of the multiple algorithms for the medical images, and the display controlling function 112 causes the terminal 50 of the user, who is the medical worker such as a physician or a radiologist, to display the screen in the display mode combining the analysis results of the multiple algorithms. Specifically, the display controlling function 112 causes the terminal 50 of the user to display on the screen, the range of the slice images to which attention needs to be paid in the slice images, based on the analysis results of the multiple algorithms. Therefore, in the medical information processing apparatus 100 according to this embodiment, when the physician wants to refer to the analysis results of the multiple algorithms, the physician does not have to spend time looking for the range to read the image, so that the efficiency of the image reading can be improved.

In addition, the medical information processing apparatus 100 according to this embodiment can support the reading of disease images because the screen in the display mode combining the analysis results of the multiple algorithms can be presented in a manner that is easy for physicians to understand. In addition, disease sites that cannot be determined by a single algorithm can be displayed by combining the results of the multiple algorithms, thereby preventing an oversight in image reading and improving the reading efficiency. In addition, while the reading range has been subjectively determined by the physician, the range of the slice images to which attention needs to be paid in the slice images can be presented based on the analysis results of the multiple algorithms.

### OTHER EMBODIMENTS

The embodiment, which has been described so far, may be implemented in a variety of different forms in addition to that described above.

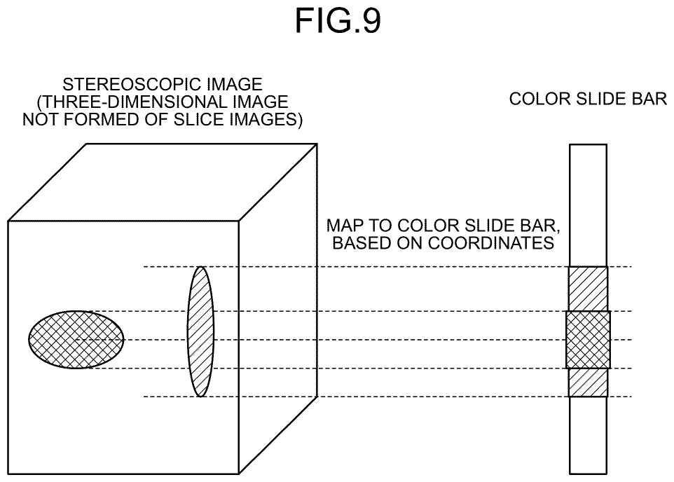

In the aforementioned embodiment, the medical image is the three-dimensional image formed of the slice images, and the display controlling function 112 causes the terminal 50 of the user to display the range of the slice images to which attention needs to be paid in the slice images, based on the analysis results of the multiple algorithms in the color slide bars on the screen. However, the embodiment is not limited to this example. In another example, as illustrated in FIG. 9, the medical image may be a three-dimensional image that is not formed of the slice images, and the display controlling function 112 may cause the terminal 50 of the user to display the range (three-dimensional region) to which attention needs to be paid in the three-dimensional image, based on the analysis results of the multiple algorithms in the color slide bars on the screen. Here, the three-dimensional image is a stereoscopic image formed of a plurality of voxels and for example, a volume rendering (VR) image resulting from a VR process of a three-dimensional region to which attention needs to be paid or a VR image of a particular organ extracted by segmentation of a three-dimensional region to which attention needs to be paid is displayed. Alternatively, a multi planer reconstruction (MPR) image of the three-dimensional region to which attention needs to be paid is displayed. The position to which attention needs to be paid in the three-dimensional image is mapped to the color slide bar, based on the information (for example, coordinates) of the three-dimensional image. In this way, the display controlling function 112 can reflect the relevant position from the stereoscopic image where the patient is photographed for the analysis results of the multiple algorithms.

For example, the medical image may be a two-dimensional image, and the display controlling function 112 may cause the terminal 50 of the user to display in the color slide bar, the range to which attention needs to be paid in the two-dimensional image, based on the analysis results of the multiple algorithms. Here, the two-dimensional image is an X-ray image such as a chest X-ray. The position to which attention needs to be paid in the two-dimensional image is mapped to the color slide bar, based on the information (for example, coordinates) of the two-dimensional image. Thus, the display controlling function 112 can reflect the relevant position from the two-dimensional image where the patient is photographed for the analysis result of each of the multiple algorithms.

For example, the acquiring function 111 and the display controlling function 112 of the medical information processing apparatus 100 according to this embodiment may be provided in separate apparatuses. In this case, the medical information processing system 1 has an apparatus with the acquiring function 111 and an apparatus with the display controlling function 112 as the functions of the medical information processing apparatus 100.

Each component of each apparatus illustrated in this embodiment is conceptual in terms of function, and does not necessarily have to be physically configured as illustrated in the drawings. In other words, the specific form of dispersion and integration of the apparatuses is not limited to that illustrated in the figure, but can be configured by functionally or physically dispersing or integrating all or some of them in arbitrary units according to various loads and usage conditions.

Furthermore, all or any part of each processing function performed by each apparatus can be realized by a CPU and a computer program analyzed and executed by the CPU, or by hardware using wired logic.

The methods described in this embodiment can also be realized by executing a computer program prepared in advance on a computer such as a personal computer or workstation. This computer program can be distributed via the Internet or other networks. This computer program can also be recorded on a computer-readable non-transitory recording medium, such as a hard disk, flexible disk (FD), CD-ROM, MO, or DVD, and executed by being read out from the recording medium by a computer.

According to at least one of the embodiments described above, the efficiency of the image reading can be improved

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to:
acquire an analysis result of each of a plurality of algorithms for a medical image; and
cause a terminal of a user to display a screen in a display mode combining the analysis results of the algorithms.

2. The medical information processing apparatus according to claim 1, wherein
the medical image is a three-dimensional image, and
the processing circuitry is configured to cause the terminal to display on the screen, a range to which attention needs to be paid in the three-dimensional image, based on the analysis result of each of the algorithms.

3. The medical information processing apparatus according to claim 2, wherein
the medical image is a three-dimensional image including a plurality of slice images,
a tool for the user to designate one of the slice images is displayed on the screen, and
the processing circuitry is configured to perform display of a slice image together with the screen, the slice image being designated by the user with the tool.

4. The medical information processing apparatus according to claim 1, wherein the processing circuitry is configured to cause the terminal to display the screen in the display mode combining the analysis results of algorithms selected by the user among the algorithms.

5. The medical information processing apparatus according to claim 1, wherein the processing circuitry is configured to cause the terminal to display the screen representing any of an OR operation result, an AND operation result, and an XOR operation result as the display mode combining the analysis results of the algorithms.

6. The medical information processing apparatus according to claim 2, wherein the processing circuitry is configured to cause the terminal to display the screen in the display mode using a color according to a score representing the analysis result of each of the algorithms.

7. The medical information processing apparatus according to claim 2, wherein the processing circuitry is configured to cause the terminal to display the screen in the display mode using a graph in which a body axis direction of a subject and a score representing the analysis result of each of the algorithms are associated.

8. The medical information processing apparatus according to claim 2, wherein the processing circuitry is configured to cause the terminal to display the screen in the display mode using a heat map in which a score representing the analysis result of a first algorithm among the algorithms and a score representing the analysis result of a second algorithm among the algorithms are associated.

9. The medical information processing apparatus according to claim 1, wherein
the medical image is a two-dimensional image, and
the processing circuitry is configured to cause the terminal to display on the screen, a range to which attention needs to be paid in the two-dimensional image, based on the analysis result of each of the algorithms.

10. A medical information processing system comprising a processing circuitry configured to:
acquire an analysis result of each of a plurality of algorithms for a medical image; and
cause a terminal of a user to display a screen in a display mode combining the analysis results of the algorithms.
